(19) United States
(12) Patent Application Publication
(10) Pub. No.: US 2015/0306288 A1
(43) Pub. Date: Oct. 29, 2015

(54) METHOD FOR PRESERVING PLACENTAL BLOOD

(71) Applicants: MACO-PHARMA, Mouvaux (FR); ETABLISSEMENT FRANCAIS DU SANG, La Plaine Saint Denis (FR)

(72) Inventors: Bruno Delorme, Marcq-en-Baroeul (FR); Zoran Ivanovic, Merignac (FR)

(73) Assignees: ETABLISSEMENT FRANCAIS DU SANG, La Plaine Saint Denis (FR); MACO PHARMA, Mouvaux (FR)

(21) Appl. No.: 14/434,768

(22) PCT Filed: Oct. 9, 2013
(86) PCT No.: PCT/FR2013/052410

§ 371 Date: Apr. 10, 2015

(30) Foreign Application Priority Data
Oct. 10, 2012 (FR) 1259684

(51) Int. Cl. A61M 1/02; A61J 1/20

(52) U.S. Cl. CPC A61M 1/0272; A61J 1/2089; A61J 1/10

(57) ABSTRACT

A method for preserving whole placental blood comprising introducing whole placental blood into an air barrier storage bag, storing said bag containing whole placental blood at a temperature of more than 0° C. and less than 40° C., so as to preserve the whole placental blood.

### BACKGROUND OF THE INVENTION

The invention relates to a method for preserving whole placental blood and a system of bags for implementing such a method.

It is applicable to short term preservation of whole placental blood, in other words blood originating from the umbilical cord and/or the placenta and before any volume reduction or cell isolation type treatment is applied to the placental blood. It is particularly applicable to whole placental blood just after removal.

Placental blood represents an attractive source for grafting hematopoietic stem cells in patients suffering from congenital or acquired hematological diseases such as cancer or leukaemia. Indeed, if there is no compatible and adult bone marrow donor, grafting of cells derived from placental blood becomes the only solution. Moreover, good grafting results can be obtained because compatibility is less rigorous than it is with adult cells. Finally, removal of placental blood is not traumatic for the mother or for the child.

Placental blood contains several types of cells including (i) cells that are not useful for the graft (red cells, platelets and granulocytes), (ii) others that can modulate the effect of the graft by immuno-modulation (lymphocytes), and finally (iii) cells that participate directly in hematopoietic reconstitution after the graft.

These latter cells are formed from a heterogeneous and largely positive population for the CD34 antigen for which two cellular sub-populations can be schematically distinguished, namely stem cells and engaged progenitors (CFC). Stem cells are very rare in the cellular content but their presence is essential for long life of the graft and for maintaining hematopoiesis in the long term.

These cells are detected depending on their capacity to induce human hematopoiesis in the bone marrow of immuno-deficient mice (SCID Repopulating Cells, SRC).

Furthermore, the engaged progenitors that are much more frequent (up to 50% of the CD34+ population in placental blood) are necessary for the early hematopoietic reconstitution phase after the graft.

In the context of creating a placental blood bank, it is essential to optimise preservation of these two sub-populations of hematopoietic cells, to optimise the effects of the placental blood graft.

Indeed, placental blood banks have been created to preserve placental blood in order to satisfy the increasing need for it. After removal of a placental blood unit (PBU) using a system of bags as disclosed in document EP-1 262 202 and after a so-called “volume reduction” step, the reduced placental blood unit is transferred into a freezing bag. The freezing bag is then placed in liquid nitrogen for long term preservation. This freezing bag is usually made from ethylene-vinyl acetate (EVA), a sterilisable material that resists cryogenic temperatures.

The EVA films are permeable to gas, and this is why they are also used frequently for manufacturing bags designed for preservation of blood platelets and cell culture, as disclosed in documents EP-A1-0 542 221 and US 2005/0032205 respectively.

It has been shown that a loss of engaged functional progenitors will occur if placental blood is not treated quickly after removal for freezing (Ivanovic et al. Transfusion, 2011).

However, in some cases, particularly when the location at which placental blood is removed, usually the hospital or the maternity, is a long way from the location at which the placental blood is treated, it is difficult to perform the treatment within 24 hours.

In order to prolong the time period between removal and treatment of placental blood, Hubel et al. proposed to add a storage solution to the placental blood collected in a bag permeable to gas, such as notably the Normosol solution, Plasmalyte A or STM-Sav (Hubel et al. Transfusion, 2003, 43, 626-632).

Similarly, documents WO 2009/121002 and WO 2009/120996 disclose the removal of placental blood in a polyvinyl chloride (PVC) bag that is permeable to air and the addition to said placental blood of the Hypothermosol® solution made by Biolife Inc. has been added. The blood is then preserved at 4° C. up to 72 hours.

Document WO 2004/032750 also discloses a method for stabilising a blood sample at ambient temperature using a receptacle containing one or several caspase inhibitors. The recipient in the form of a tube may be permeable, semi-permeable or impermeable to oxygen. This method can stabilise blood for a few hours until it is analysed, but it is not suitable for medium term preservation, in other words for more than a day, of whole placental blood in view of its storage in the bank.

### BRIEF DESCRIPTION OF THE DRAWINGS

The invention is aimed at preserving placental blood in the short term before treatment, while maintaining viability and functionality of at least the stem cells and hematopoietic progenitors of a placental blood unit. Moreover, the invention discloses a method in which the viability and functionality of stem cells and hematopoietic progenitors are maintained throughout the treatment of the placental blood unit, from collection until grafting.

To achieve this, according to a first aspect, the invention relates to a method for preservation of whole placental blood including steps to:
introduce whole placental blood into an air barrier storage bag,
store said bag containing whole placental blood at a temperature of more than 0° C. and less than 40° C., so as to preserve the whole placental blood.

According to a second aspect, the invention relates to a system of bags for implementing a method according to the first aspect, said system comprising:
a placental blood collection bag in fluid communication, through a first tube associated with an inlet orifice of the collection bag, with at least one draw-off needle,
an air barrier storage bag, said bag being in fluid communication or intended to be put into fluid communication with the collection bag so that it can store the placental blood.

Other purposes and advantages will become apparent in the following description.

FIG. 1 shows a collection bag in a system of bags for the implementation of a method according to a particular embodiment of the invention.

FIG. 2 shows a storage bag in a system of bags intended to be put into fluid communication with the collection bag in FIG. 1.

FIG. 3 shows a system of bags obtained after connection of the collection bag in FIG. 1 and the storage bag in FIG. 2.

FIG. 4 shows a graph illustrating the preservation of hematopoietic progenitors (CFC) stored at 4° C. for 3 days under different storage conditions.

FIG. 5 shows a graph illustrating the preservation of cells positive to the CD34 marker (CD34+) stored at 4° C. for 3 days under different storage conditions.

FIG. 6 shows a graph illustrating the viability of CFCs stored at 4° C., between 0 and 14 days, with or without the presence of the preservation solution MC01.

FIG. 7 shows a graph illustrating the expansion capacity of CD34+ and CFC cells, preserved at 4° C., between 0 and 48 hours, with or without the presence of the preservation solution MC01.

FIG. 8 shows a graph illustrating the preservation of total nucleated cells (TNC), mononucleated cells (MNC), CD34+ cells (viable) and CFC cells stored at 4° C. for 3 days in bags permeable to air (PVC) and not permeable to air (TriC).

FIG. 9 shows a graph illustrating the preservation of TNC, MNC, CD34+ and CFC cells stored in air barrier bags (TriC) at 4° C. for 3 days, with or without the presence of the solution MC01.

FIG. 10 shows a graph illustrating the preservation of TNC, MNC, CD34+ and CFC cells stored in PVC bags at 4° C. for 3 days without a preservation solution (current situation for the PBU bank) or in air barrier bags (TriC) with a preservation solution (MCO1).

FIG. 11 shows a graph illustrating the preservation of viable CD34+ cells and CFC cells during the treatment of placental blood units stored in air barrier bags (TriC), with or without a preservation solution (MC01): at D0, at D3 after storage for 3 days at 4° C., after volume reduction using the “Sepax” (AS) method, after thawing (AD) and at 6 hours after thawing (AD+6 h).

FIG. 12 shows a graph illustrating the number of CFC/femur (log) after grafting a placental blood unit stored under routine conditions or according to the method of the invention.

### DETAILED DESCRIPTION

At the present time, the umbilical cord blood or placental blood must be treated within 24 hours after collection. If it were possible to postpone the treatment of placental blood until 72 hours after its collection, it would be possible to collect placental blood in many maternity centres and have centralised banks that could thus potentially be a long way away from the collection sites. This would also enable private banks to propose collection over longer distances from their banking centre.

Thus, and according to a first aspect, the invention discloses a method for the preservation of whole placental blood including steps to:
introduce whole placental blood into an air barrier storage bag,
store said bag containing the whole placental blood at a temperature above 0° C. and below 40° C., so as to preserve the whole placental blood.

This preservation method applies to whole placental blood, in other words blood originating directly from an umbilical cord and/or a placenta. This blood has not been treated, other than possibly by the addition of an anticoagulant, and in particular no volume reduction treatment has been carried out on it, for example by sedimentation of red cells and/or centrifuging. Nor has a freezing step been carried out on the blood. In this method, the whole placental blood is kept in a non-frozen liquid state. The method does not apply either to purified cells and/or cells isolated from umbilical cord blood, for example by density gradient.

There are essentially two methods of removing placental blood. According to a first practice, the placental blood is removed during delivery so that the mother's contractions will help the blood flow into a bag.

According to a second practice, the procedure starts after the placenta has been expelled and, after separation of the cord from the baby, the placenta is placed on a work table from which the cord is allowed to hang to collect placental blood by gravity.

Regardless of the method used, the volume of placental blood derived from a removal is called a “placental blood unit” (PBU). The volume of one PBU is comprised between 50 and 200 ml, and particularly between 80 and 120 ml.

The method according to the invention is applicable to placental blood once it has been extracted from the umbilical cord and/or the placenta. In particular, the method is applicable within 24 hours after the placental blood has been removed. After 24 hours, the loss of cells of interest becomes too high.

The cells of interest are particularly hematopoietic stem cells and hematopoietic engaged progenitors that are useful for hematopoietic reconstitutions after a placental blood graft.

The preservation method according to the invention is intended particularly to preserve hematopoietic stem cells and hematopoietic engaged progenitors contained in placental blood, in other words it preserves the viability and functionality of these cells, as shown in the following examples.

In particular, after 3 days of storage, the method can give a content of viable CD34+ hematopoietic stem cells equal to at least 80%, particularly at least 90% and even more particularly close to 100%, in relation to the number of viable CD34+ cells in the placental blood unit immediately after removal.

Thus, after 3 days of storage, the method can give a content of viable hematopoietic progenitors (CFC) equal to at least 75% and particularly at least 80% and even more particularly at least 90% in relation to the number of viable progenitors in the placental blood unit immediately after removal.

The method thus consists of introducing whole placental blood into an air barrier storage bag and storing said bag containing the whole placental blood.

The storage bag is adapted to contain at least one sample of placental blood. In particular, the capacity of the storage bag is comprised between 50 mL and 1 L, and particularly comprised between 100 mL and 500 mL. In particular, the capacity of the storage bag is 500 mL.

In particular, the air barrier storage bag is a bag comprising an envelope for storing the placental blood. The envelope is made with an air barrier film and/or is arranged in a packaging made with an air barrier film. The air barrier film is an oxygen barrier film or an oxygen and carbon dioxide barrier film.

When the bag comprises an air barrier packaging, the envelope of the bag may be made from a film permeable to air such as PVC.

An “oxygen barrier film” is a film with permeability to oxygen of less than 10 cm³/m²·day·atm. Permeability to oxygen is determined according to ASTM standard D-3985.

A “carbon dioxide barrier film” is a film with permeability to carbon dioxide of less than 15 cm³/m²·day·atm. The permeability to carbon dioxide is determined according to ASTM standard F-2476.

The air barrier film comprises an air barrier polymer such as ethylene vinyl alcohol (EVOH) copolymer, vinylidene chloride copolymer, polyvinyl alcohol, polyacrylonitrile or polyamide.

The air barrier film is particularly a film comprising an ethylene vinyl alcohol (EVOH) copolymer.

In particular, the air barrier film has a multi-layer structure such as a three-layer structure in which the central layer is made from an air barrier material. The central layer is sandwiched between two layers made from another material such as polyolefin, and particularly polyethylene, polypropylene or an ethylene-olefin copolymer.

For example, the film is a three-layer film made of ethylene vinyl acetate/ethylene vinyl alcohol/ethylene vinyl acetate (EVA/EVOH/EVA).

This air barrier film made of EVA/EVOH/EVA is special in that it gives good resistance to steam sterilisation techniques (heating to 121° C. for 15-20 minutes) and is biocompatible with the contents of the bag.

According to one particular embodiment, the thickness of the film forming the envelope of the bag is comprised between 0.20 and 0.80 mm, and particularly 0.50 mm.

According to another embodiment, the envelope of the storage bag is made from a film for which the permeability to oxygen is of the order of 2.2 cm²/m²·day·atm.

The envelope of the storage bag is made from a film with a permeability to carbon dioxide of the order of 6 cm³/m²·day·atm.

The concentration of physiological oxygen in placental blood varies from 1.1 to 4% (Kotaska et al., J Clin Lab Anal 24: 300-4). The use of an air barrier bag can limit oxygen added to placental blood, so that it remains close to the physiological oxygen concentration.

This would preserve the functionality of cells of interest in placental blood, and particularly hematopoietic stem cells and engaged progenitors.

Advantageously, the placental blood in added into the storage bag within 24 hours after removal, before the loss of viability of cells of interest becomes too high.

More particularly, placental blood is introduced into a storage bag immediately after removal, in order to minimise oxygenation of placental blood.

The storage bag containing the placental blood is stored at a temperature above 0° C. and below 40° C., and particularly at ambient temperature or at 4° C. Placental blood is not frozen and remains in the liquid state.

According to one particular method, the bag is stored in the short term, in other words between 1 and 14 days, and particularly between 1 and 5 days and more particularly between 1 and 3 days.

According to one particular form, the preservation method includes, before the storage step, a step for adding placental blood to an anticoagulant solution.

For example, the anticoagulant solution may be an acid citrate dextrose solution (ACD) or a citrate phosphate dextrose (CPD) solution.

The quantity of placental blood derived from removal of blood from the cord varies between 50 and 200 ml on average. The quantity of anticoagulant solution to be added is about 20-30 ml.

According to a first variant, the placental blood is collected in a collection bag that contains the anticoagulant solution or into which the anticoagulant solution is transferred, such that the mix of placental blood and anticoagulant solution is introduced into the storage bag.

As a variant, the placental blood is introduced into the storage bag that contains the anticoagulant solution or into which the anticoagulant solution is transferred.

Advantageously, before the storage step, the method includes a step to add placental blood with a preservation solution. In this case, the air barrier storage bag that will be stored contains whole placental blood and a preservation solution.

The preservation solution is a solution different from the anticoagulant solution.

Combination of an air barrier bag and a preservation solution can optimise storage conditions. Under these conditions, the cells of interest are preserved at about 100% of the cells present immediately after the removal of placental blood.

In one particular embodiment, the preservation solution includes a solution of electrolytes, a cell or tissue preservation solution or a culture medium.

For example, the preservation solution comprises a solution of electrolytes. The solution of electrolytes comprises for example sodium, potassium, calcium, chloride, zinc, iron and/or magnesium ions.

Such an electrolyte solution may for example be the Plasmalyte-A®, Normosol-R® or the Ringer or Ringer lactate solution.

As a variant, the preservation solution also comprises a buffer and/or one or several antioxidants.

The buffer may for example be chosen from among physiological buffers (sulphate, phosphate or carbonate) or synthetic buffers (HEPES).

Examples of antioxidants are free radical traps; iron chelators such as deferoxamine; vitamin E, vitamin C or sodium erythorbate; and thiolated derivatives such as N-acetylcysteine, glutathion or reduced glutathion.

According to one variant, the preservation solution comprises one or several oncotic agents in addition to the solution of electrolytes. An oncotic agent is a molecule applying oncotic pressure to avoid cellular oedema. The oncotic agent may be an impermeabilising agent such as raffinose, sucrose or mannitol and/or a colloid such as hydroxyethyl starch, albumin or polyethylene glycol.

In particular, the oncotic agent is albumin or an albumin substitute. For example, the preservation solution comprises a solution of electrolytes, albumin and a buffer.

In another example, the preservation solution comprises a preservation solution of cells and tissues. For example, such a solution comprises a solution of electrolytes, an oncotic agent and a buffer.

As a variant, the cell or tissue preservation solution also comprises complements such as adenosine as an energy substrate and/or one or several antioxidants.

For example, such a preservation solution may be the Hypothermosol® solution made by Biolife Solutions.

As a variant, the preservation solution comprises amino acids, vitamins and glucose in addition to the solution of electrolytes.

For example, the preservation solution comprises a basal culture medium. The basal medium is a medium comprising essentially a solution of electrolytes, vitamins, amino acids, glucose for its energy supply and a buffer.

For illustration purposes, the basal medium is DMEM (Dulbecco's Modified Eagle's Medium), MEM (Minimal Essential Medium), RPMI 1640, F-10, F-12, αMEM (α Minimal Essential Medium) and IMDM (Iscove's Modified Dulbecco's Medium).

In particular, the basal culture medium also comprises complements particularly such as non-essential amino acids, minerals, trace elements and/or
insulin or an insulin substitute composed of a zinc salt,
transferrin or a transferrin substitute as an iron chelator,
albumin or an albumin substitute such as polyethylene glycol (PEG),
lipids and/or fatty acids and/or
one or more antioxidants.

In one particular form, the preservation solution has no serum.

One particularly advantageous preservation solution is the MC01 culture medium developed by Maco Pharma and marketed under the name HP02.

This medium comprises:
a commercial basal medium comprising electrolytes, vitamins, amino acids, glucose and sodium pyruvate for their energy supply, a HEPES buffer and phenol red as the pH indicator,
basal complements particularly trace elements,
insoluble lipids,
recombining human insulin (1-100 mg/L),
human albumin (0.1-0.8%),
iron gluconate (II) (50-1000 mg/L),
antioxidants,
specific complements for expansion of these cells such as nucleosides (0.1-10 mg/L).

In particular, the ratio between the preservation solution and placental blood in the bag is comprised between 1:0.5 and 1:2, and advantageously 1:2. The quantity of preservation solution must not be too high so that it does not have a prejudicial effect on other treatment steps of the placental blood unit, and particularly the volume reduction step. For example, the quantity of the added preservation solution for a standard PBU is about 50 mL.

According to one particular method, the preservation solution is added to the placental blood before the storage step.

According to a first variant, the placental blood is collected in a collection bag that contains the preservation solution or into which the preservation solution is transferred, such that the mix of placental blood and the preservation solution is introduced into the storage bag.

As a variant, the placental blood is introduced into the storage bag that contains the preservation solution or into which the preservation solution is transferred.

When the method comprises the addition of an anticoagulant solution and a preservation solution, it is advantageous to:
collect the placental blood in a collection bag containing the anticoagulant solution,
add the preservation solution to the anticoagulated placental blood,
introduce the anticoagulated placental blood to which the preservation solution has been added into the storage bag.

Even more advantageously, the preservation solution is initially placed in the air barrier storage bag, so that the preservation solution can be stored before use.

After preservation of the placental blood unit according to the method disclosed above, the placental blood unit is treated in a normal manner.

For example, the placental blood unit is subjected to a volume reduction or miniaturisation step, particularly using the Sepax instrument (Zingsem J et al., Transfusion, 2003; 43:806-13).

A cryoprotectant agent such as DMSO (dimethyl sulfoxide) is added to the placental blood unit so that it can be frozen and preserved in the long term, in other words up to several months, at highly negative temperatures, particularly in liquid nitrogen at −196° C.

When a graft is necessary, the placental blood unit is thawed, possibly washed to eliminate the cryoprotectant agent (DMSO), and then injected to the patient.

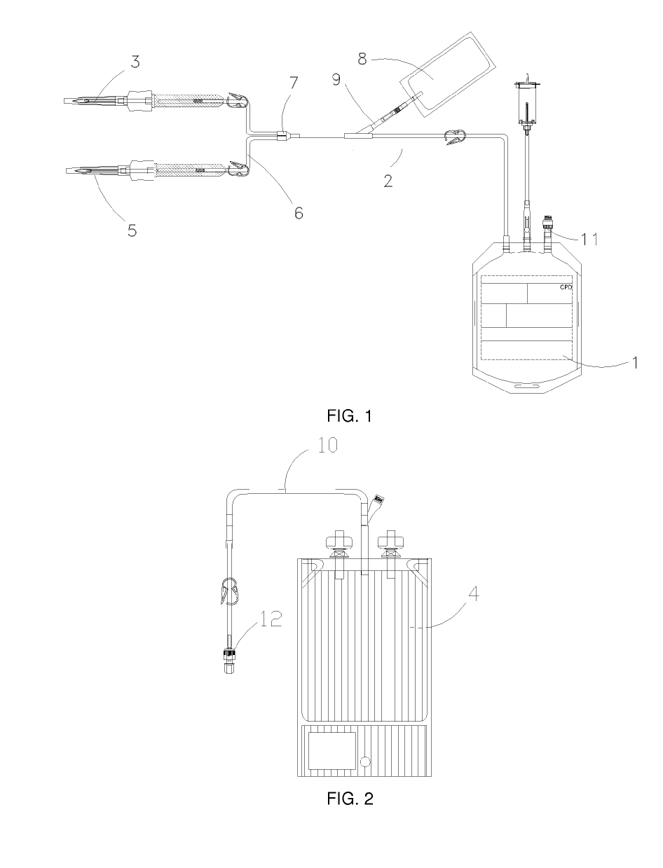

According to another aspect and with reference to FIGS. 1 to 3, the invention relates to a system of bags for implementation of a method according to the first aspect of the invention comprising:
a placental blood collection bag 1 in fluid communication, through a first tube 2 associated with an inlet orifice of the collection bag 1, with at least one draw-off needle 3,
an air barrier storage bag 4, said bag being in fluid communication or intended to be brought into fluid communication with the collection bag 1, so that the whole placental blood can be stored.

In particular, the collection bag 1 is made from polyvinyl chloride (PVC). The collection bag is connected through a first tube 2 to a draw-off needle 3.

In another embodiment, the system of bags comprises a second draw-off needle 5 to prick the umbilical cord at another location in order to retrieve a maximum amount of placental blood.

In this case, the second draw-off needle 5 is in fluid communication through a second tube 6 connected to the first tube 2 at a Y connector 7.

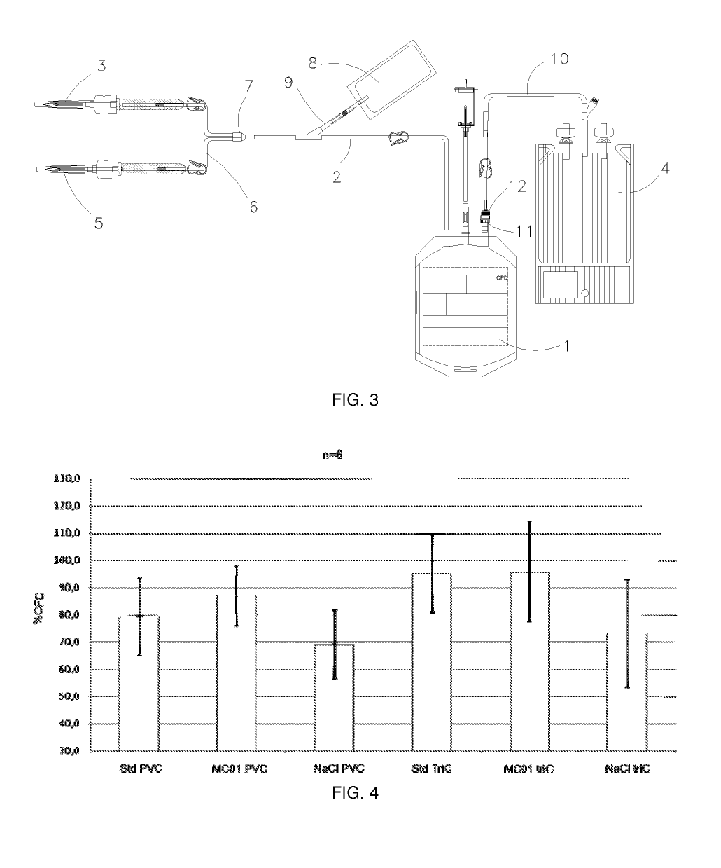

In FIGS. 1 and 3, the collection bag 1 contains a CPD or ACD type anticoagulant solution in order to avoid coagulation of the collected placental blood.

Advantageously, the system of bags comprises a secondary bag 8 containing an anticoagulant solution that may be different or identical to that of the collection bag 1. This secondary bag 8 is in fluid communication with the collection bag 1 through a third tube 9 connected onto the first tube 2.

The anticoagulant solution present in this secondary bag 8 is used to partly rinse the first tube 2 at the end of collection of placental blood in order to retrieve a maximum amount of placental blood. The volume of anticoagulant solution present in this bag is about 8 ml.

The air barrier storage bag 4 is particularly the bag disclosed above with reference to the preservation method.

Advantageously, the storage bag 4 contains a preservation solution like that defined above.

In one particular form shown in FIG. 3, the storage bag 4 is in fluid communication with the collection bag 1 through a tube 10.

If the collection bag 1 and the storage bag 4 are connected at the manufacturing stage, the system of bags forms a closed system.

As a variant, the storage bag 4 is designed to be brought into fluid communication with the collection bag. In this case, the collection bag 1 and the storage bag 4 are provided with complementary connection means 11, 12.

For example, the collection bag 1 is provided with a female Luer type connector 11 at one of its orifices, and the storage bag 4 is provided with a tube 10 at one of its access orifices, the end of which that is not connected to the storage bag is fitted with a male Luer type connector 12.

The tubes may be fitted with clamps so that the fluid flow between the different bags can be opened or closed.

### EXAMPLES

Experiments have been carried out to study the preservation of progenitors and hematopoietic stem cells during preservation of placental blood in air barrier bags. As a complement or alternately, the effect of adding a preservation solution has also been tested.

In these examples, the preservation solution is the MC01 culture medium specifically developed by Maco Pharma. Placental blood is anticoagulated, in order words it contains an anticoagulant (CPD). Air barrier bags are made with a three-layer EVA/EVOH/EVA film with permeability to oxygen of the order of 2.2 cm²/m²·day·atm and a permeability to carbon dioxide of the order of 6 cm³/m²·day·atm (triC bag).

### Example 1

### “Miniature” Tests

### Example 1.1

### Effect of Using Air Barrier Bags

A blood sample (8 ml) originating from a placental blood unit (PBU) of less than 24 hours after the birth was transferred into a bag permeable to air (PVC) or an air barrier bag (TriC). The placental blood is then put in a refrigerator at +4° C. and stored for 3 days.

Under certain conditions, a preservation solution is added to the placental blood. The proportion of the preservation solution/blood is 1:2 (4 ml of preservation solution+8 ml of placental blood). This proportion is considered to be conducive to the development that is aimed at a clinical application.

Placental blood was then mixed with the preservation solution on arrival at the laboratory (<24 h) and preserved for 3 days at +4° C.

The series of experiments was carried out under the following 6 conditions:

Sdt PVC: 8 ml of placental blood in a PVC bag

MC01 PVC: 8 ml of placental blood+4 ml of MC01 in a PVC bag

NaCI PVC: 8 ml of placental blood+4 ml NaCl in a PVC bag

Sdt TriC: 8 ml of placental blood in an air barrier bag

MC01 TriC: 8 ml of placental blood+4 ml of MC01 solution in an air barrier bag

NaCl TriC: 8 ml of placental blood+4 ml NaCl in an air barrier bag.

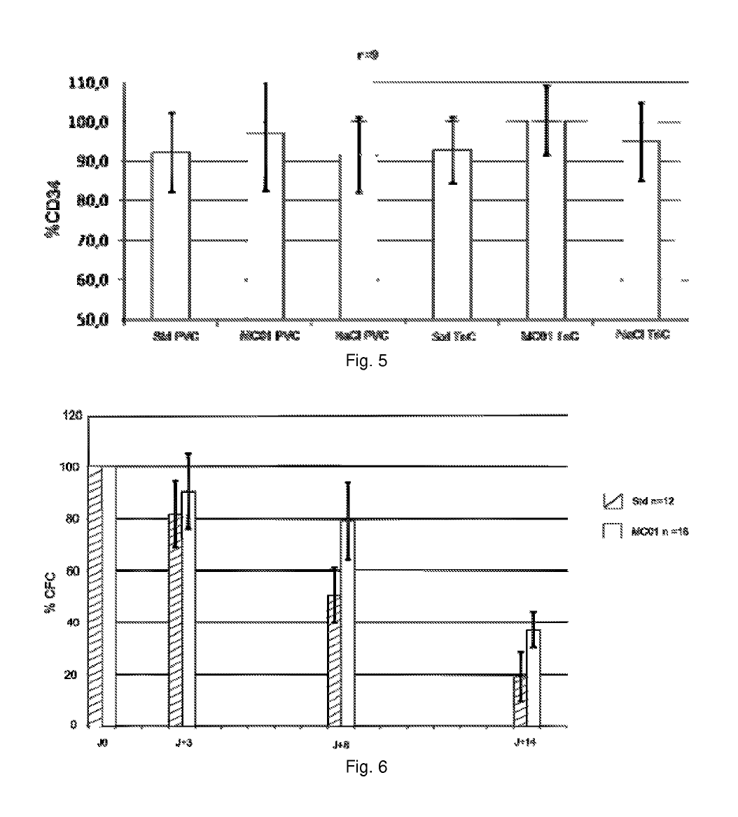

The number of total nucleated cells (TNC), mononucleated cells (MNC), CD34+ cells and CFC cells was determined. Only the most illustrative data concerning CFC and CD34+ cells are presented (FIGS. 4 and 5).

This series of manipulations showed a positive effect of the use of air barrier bags. Indeed, the percentage of CD34+ and CFC cells in PBUs stored in air barrier bags is always greater than that obtained in PVC bags that are permeable to air. In particular, the effect of storing placental blood in air barrier bags on CFC cells, for which the percentage is higher than 90%, is particularly advantageous.

Similarly, it is noted that the addition of a preservation solution has a positive effect that can be seen on CD34+ cells.

Finally, the preservation of progenitors and CD34+ cells in an air barrier bag in combination with a preservation solution is almost total, in other words close to 100%.

### Example 1.2

### Effect of the Preservation Solution

Another series of experiments was carried out in 30 ml PVC bags permeable to air. Blood originating from a placental blood unit (PBU) of less than 24 h after birth was mixed with the preservation solution (10 ml+10 ml). For control (Std), the placental blood does not contain any preservation solution. Placental blood is then put in a refrigerator at +4° C. and is stored for 14 days. The numbers of total cells, cells positive to the CD34 marker (CD34+) and CFC cells are determined at D0, 3, 8 and 14.

FIG. 6 shows the variation in the CFC percentage in comparison with D0 during this storage (D+3, D+8, D+14). A positive effect of the preservation solution on maintaining these functional progenitors at +4° C. can be seen.

The capacity of CD34+ cells preserved for two days (48 hours) with or without a preservation solution to amplify themselves ex vivo was tested under the same storage conditions. The expansion method used is described in Ivanovic et al, Cell Transplant 20: 1453-63 2011.

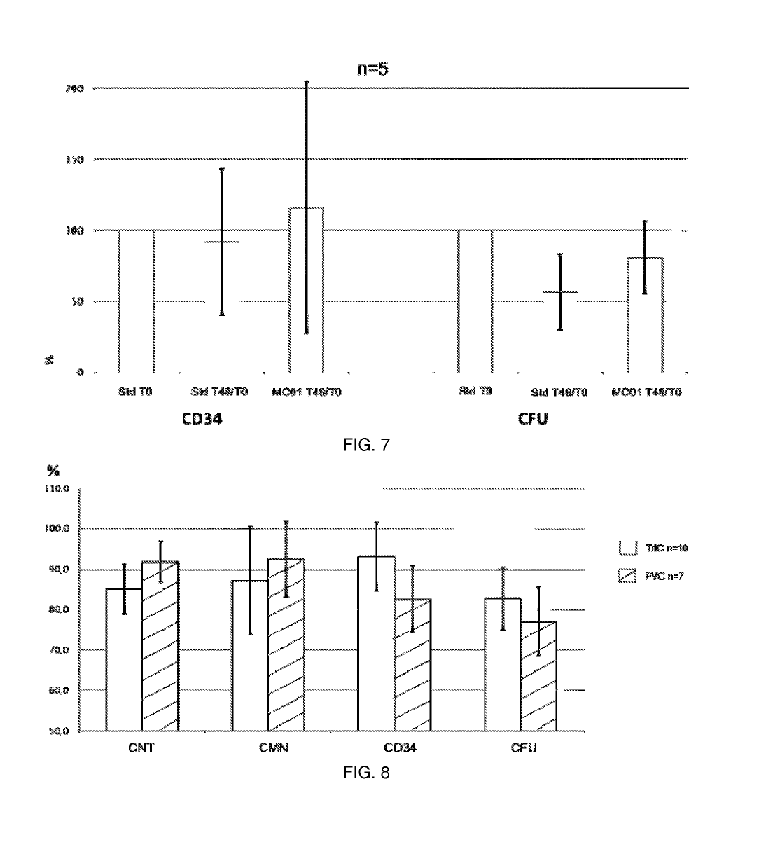

The results (FIG. 7) show that, without the preservation solution, the expansion capacity relative to T0 reduces to 50% at 48 h, whereas in the presence of a preservation solution (MC01), it is maintained and is significantly much higher than it is for the condition without the preservation solution (Std).

### Example 3

### “Full Scale” Tests

“Full scale” experiments, in other words with a standard PBU with a volume of between 80 and 120 ml, were carried out during which placental blood is preserved in bags permeable to air (PVC) or in air barrier bags (TriC) for 3 days (FIG. 8).

In this case also, the cells of interest (CD34+ and CFC) are preserved better in an air barrier bag than in a PVC bag that is permeable to air. This effect is more marked on the CD34+ cells than on CFC cells (which is contrary to the results for “miniature” tests).

Thus, this series of experiments shows that storage of whole placental blood in an air barrier bag can preserve cells of interest for at least 3 days at 4° C.

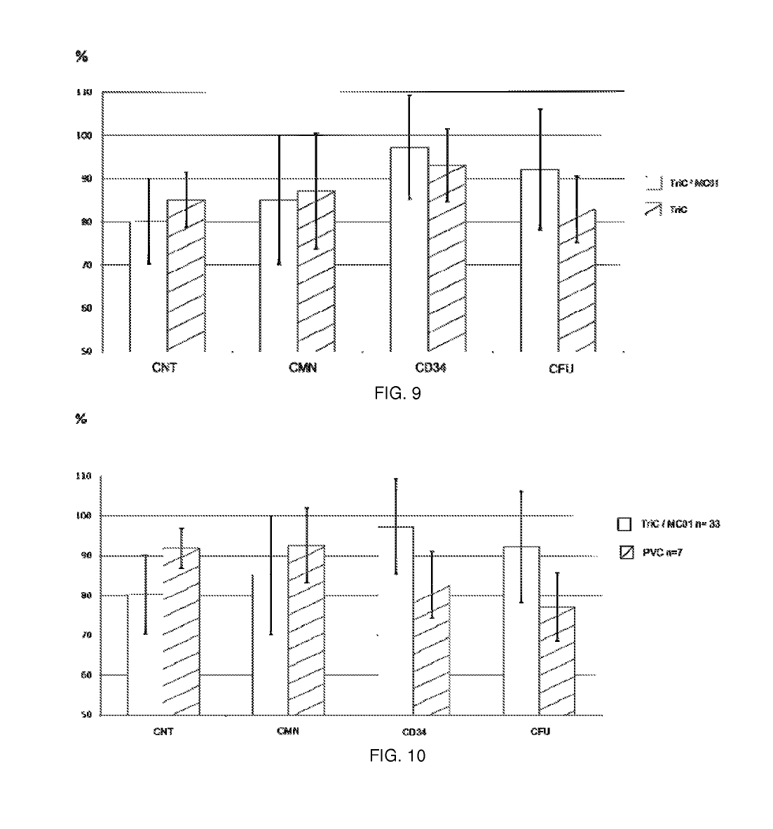

Preserving these cells for 3 days in an air barrier bag with or without a preservation solution (50 ml) was also tested. This series of manipulations shows that the presence of a preservation solution further improves the preservation of cells of interest, and particularly CFC clonogenic progenitors (FIG. 9).

FIG. 10 compares the preservation of placental blood at +4° C. for 3 days under routine conditions (PVC bags permeable to air without preservation solution) and under the more advantageous conditions of the invention (air barrier bags and preservation solution).

The results of this series of experiments can be seen clearly: the combination of an air barrier bag and the preservation solution improves preservation of CFC cells and CD34+ cells to make it ⅓ higher than in standard bags without a preservation solution. This unequivocally suggests the advantage of using the preservation solution with air barrier bags to preserve functional progenitors.

Therefore this series of experiments shows that the principle of protection of cells by prevention/limitation of their hyperoxygenation can take place in placental blood at +4° C. without prior separation of cells.

Furthermore, the combination of air barrier bags and a preservation solution can preserve practically all hematopoietic progenitors for 3 days at +4° C., whereas with bags permeable to air and without a preservation solution, preservation is about 85% for CD34+ and 75% for clonogenic progenitors.

### Example 4

### Pre-Clinical Study

To evaluate the preservation method in a situation as close as possible to the reality of routine use, a study was undertaken to study the absolute number of progenitors (CFC) and the activity of hematopoietic stem cells (SRC) before and after each placental blood treatment step. This treatment includes:
storage at 3 days (D3),
reduction in volume (also called “miniaturisation”) using the Sepax technology (AS),
controlled freezing (Bioarchive technology),
thawing (AD) and
the functional state of cells 6 hours after thawing (AD+6 h).

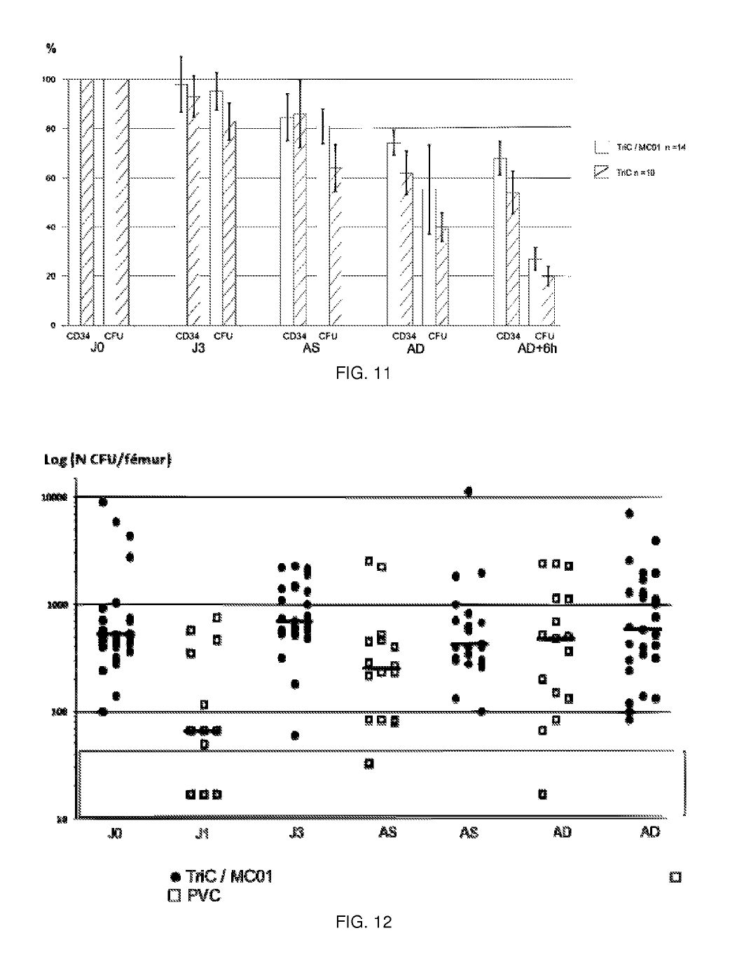

The comparison of the preservation of CD34+ and CFC cells after 3 days of storage, such as after each step, in an air barrier bag, with and without the addition of 50 ml of preservation solution, mentioned above, is shown on FIG. 11.

It is obvious that the combination of air barrier bags and the preservation solution makes it possible to preserve all CD34+ cells and hematopoietic progenitors (the percentage of hematopoietic progenitors with no medium is about 80% after 3 days storage). This advantage that relates to preservation of progenitors (CFC) is well maintained after miniaturisation by Sepax and after thawing of the samples.

Therefore these results show that biologically, the method according to the invention is compatible with an addition of medium in the maternity. This means that with such a method, hematopoietic progenitors can be better preserved at +4° C. for up to 72 hours after removal, which should enable transport of PBUs to the bank regardless of the distance between the maternity and the bank. It also confirms that the advantage obtained is maintained throughout the cryopreservation process of placental blood until the product is thawed.

### Example 5

### Study of the Preservation of Stem Cells Using the In Vivo Approach (on NOG/Scid Mice)

Unlike engaged progenitors that participate in the first wave of hematopoietic reconstitutions after grafting without being able to provide a long-term effect, stem cells take over slightly later but their effect is long term and therefore assures that the graft lasts.

The experimental approach to highlight these stem cells includes an in vivo test: graft of immuno-deficient mice (that do not reject human cells). NOG/Scid mice are used. These mice have a better graft threshold than the NOD/Scid mice used previously.

The mice are briefly conditioned with busulfan. Human cells are injected 2 days after conditioning, and the resulting graft is studied 8 weeks after. Each mouse received a dose of CD34+ cells originating from the same fraction of placental blood unit as that containing 1000 CD34+ cells at T0. The results are thus directly comparable without any conversion calculation.

After sacrificing the mice 8 weeks after the graft, the presence of human origin cells positive to CD45, CD33 and CD19 in the bone marrow is studied. The content of hematopoietic progenitors (CFC) with human origin was also studied by implantation of these cells (cell suspension recovered from the bone marrow of mice) in methylcellulose cultures with growth factors specific to human cells. For simplicity reasons and due to the functional nature of the latter test, only results based on this detection of human progenitors in the bone marrow of mice are shown.

These results (FIG. 12) show that SRCs are fully preserved for 3 days in an air barrier bag with the preservation solution throughout the treatment of PBUs until they are thawed. This preservation appears to be better than that obtained with a miniaturised PBU frozen after 24 h in a bag permeable to air (PVC) without a preservation solution (condition suitable for routine).

Therefore these results show that the approach consisting of using air barrier bags and a preservation solution can extend the preservation of progenitors and hematopoietic stem cells of placental blood to 72 h (3 days) without any loss of activity of these critical populations in comparison to a simple 24 hours storage in PVC bags, in other words according to current routine.

1-23. (canceled)

24. A method for preserving whole placental blood comprising:
introducing whole placental blood into an air barrier storage bag,
storing said bag containing whole placental blood at a temperature of more than 0° C. and less than 40° C., so as to preserve the whole placental blood.

25. The method according to claim 24, further comprising: preserving hematopoietic stem cells and hematopoietic engaged progenitors contained in placental blood.

26. The method according to claim 24, wherein placental blood is introduced into the storage bag within 24 hours after removal.

27. The method according to claim 24, further comprising: before the storage step, adding placental blood to an anticoagulant solution.

28. The method according to claim 27, wherein the anticoagulant solution is at least one of an acid citrate dextrose (ACD) solution and a citrate phosphate dextrose (CPD) solution.

29. The method according to claim 24, further comprising: before the storage step, the method includes a step to add placental blood with a preservation solution.

30. The method according to claim 29, wherein the preservation solution includes a solution of electrolytes.

31. The method according to claim 30, wherein the preservation solution comprises one or several oncotic agents.

32. The method according to claim 31, wherein the oncotic agent is albumin or an albumin substitute.

33. The method according to claim 29, wherein the preservation solution comprises amino acids, vitamins and glucose.

34. The method according to claim 29, wherein the preservation solution comprises one or several antioxidants.

35. The method according to claim 29, wherein a ratio between the preservation solution and placental blood in the bag is comprised between 1:0.5 and 1:2.

36. The method according to claim 24, wherein the bag is stored for between 1 and 14 days.

37. The method according to claim 24 wherein said storage bag comprises an envelope for storing placental blood, said envelope being made with an air barrier film and/or being arranged in a packaging made with an air barrier film.

38. The method according to claim 40, wherein the air barrier film has a three-layer structure in which the central layer made from an air barrier material is sandwiched between two layers made from another material.

39. The method according to claim 40, wherein the air barrier film is a three-layer film made of ethylene vinyl acetate/ethylene vinyl alcohol/ethylene vinyl acetate (EVA/EVOH/EVA).

40. A system of bags comprising:
a placental blood collection bag in fluid communication, through a first tube associated with an inlet orifice of the collection bag, with at least one draw-off needle,
an air barrier storage bag, said bag being in fluid communication or intended to be brought into fluid communication with the collection bag, so that the placental blood can be stored.

41. The system of bags according to claim 40, wherein the collection bag contains an anticoagulant.

42. The system of bags according to claim 40, wherein the collection bag contains a preservation solution.

43. The system of bags according to claim 40, wherein the collection bag comprises an envelope for storing placental blood, said envelope being made with an air barrier film and/or being arranged in a packaging made with an air barrier film.

44. The system of bags according to claim 43, wherein the air barrier film has a three-layer structure in which the central layer made from an air barrier material is sandwiched between two layers made from another material.

45. The system of bags according to claim 43, wherein the air barrier film is a three-layer film made of ethylene vinyl acetate/ethylene vinyl alcohol/ethylene vinyl acetate (EVA/EVOH/EVA).

46. The system of bags according to claim 40, wherein the storage bag is made with a film with permeability to oxygen of less than 10 cm³/m²·day·atm.
